(19) United States
(12) Patent Application Publication
(10) Pub. No.: US 2024/0197197 A1
(43) Pub. Date: Jun. 20, 2024

(54) MRI BASED SYSTEM AND METHOD FOR DETECTING IRON-RELATED ENTITIES IN A REGION OF INTEREST

(71) Applicant: YISSUM RESEARCH DEVELOPMENT COMPANY OF THE HEBREW UNIVERSITY OF JERUSALEM LTD., Jerusalem (IL)

(72) Inventors: Aviv MEZER, Jerusalem (IL); Shir FILO, Jerusalem (IL)

(73) Assignee: YISSUM RESEARCH DEVELOPMENT COMPANY OF THE HEBREW UNIVERSITY OF JERUSALEM LTD., Jerusalem (IL)

(21) Appl. No.: 18/286,618

(22) PCT Filed: Apr. 13, 2022
(86) PCT No.: PCT/IL2022/050383

§ 371 Date: Oct. 12, 2023

Related U.S. Application Data
(60) Provisional application No. 63/174,209, filed on Apr. 13, 2021.

(51) Int. Cl. A61B 5/055; A61B 5/00; G01R 33/50

(52) U.S. Cl. CPC A61B 5/055; A61B 5/0037; A61B 5/0042; G01R 33/50

(57) ABSTRACT

A method of detecting iron-related entities in a region of interest, is disclosed. The method may comprise: receiving at least two Magnetic Resonance Imaging (MRI) signals from each of at least two different locations in the region of interest; calculating at least four MRI parameters from the MRI signals; and determining a presence of at least two different iron-related entities in the region of interest based on the at least four MRI parameters.

### FIELD OF THE INVENTION

The present invention relates generally to methods of detecting iron-related entities in a region of interest. More specifically, the present invention relates to using an MRI-based method for detecting iron-related entities in a region of interest.

### BACKGROUND OF THE INVENTION

Iron is the most abundant trace element in the human body. It enters the brain across the blood-brain-barrier and participates in many biological processes such as oxygen transport, cellular metabolism, myelin formation, and the synthesis of neurotransmitters. Therefore, strict iron regulation is essential for maintaining normal physiological brain function. The two iron compounds most involved in iron regulation are transferrin and ferritin. Transferrin is the main iron transport protein, which carries iron from the blood into brain tissue. Ferritin is the major iron storage protein, which stores excess iron atoms not immediately engaged in metabolic activities. These iron compounds are distributed inhomogeneously among cell types and across the brain.

Accumulation of iron and disturbances in the regional distribution of specific iron compounds were identified in aging and neurodegenerative diseases such as Parkinson's disease (PD), Alzheimer's disease (AD), and Multiple Sclerosis. When iron concentrations exceed the capacity of iron-binding proteins this can lead to oxidative stress and cellular damage 10. For example, while the iron was found to accumulate in the brains of elderly controls, PD, and AD patients, the ratio of transferrin to iron, which reflects iron mobilization capacity, was shown to differ between elderly controls and either PD or AD patients, in a brain region-dependent manner.

Disturbances in the homeostasis of iron compounds, including ferritin and transferrin, were also reported in cancer cells. For instance, tumor cells proliferation requires modulated expression of proteins involved in iron uptake. In addition, iron has multiple regulatory effects on the immune system. Therefore, the availability of iron to tumor cells may affect their survival, growth rate, and the course of the disease. Meningioma is a very common brain tumor, which was shown to contain a higher concentration of ferrimagnetic particles and abnormal expression of iron-related genes compared to non-pathological tissue. This evidence implies there are differences in iron metabolism between meningioma tumors and normal brain tissue. The diagnosis of brain tumors and their delineation from the surrounding non-pathological tissue is routinely performed based on contrast-enhanced MRI, which requires injection of gadolinium (Gd)-based agents 18. Depositions of gadolinium found in the brain have recently renewed concerns about the long-term safety of Gd-based agents, highlighting the need for non-invasive MRI techniques that can serve as alternatives.

The extensive implications of impaired iron homeostasis in aging, neurodegeneration, and carcinogenesis suggest that assessment of different iron compounds in the living brain would be highly valuable for diagnosis, therapeutic monitoring, and understanding pathogenesis of diseases 4. Iron's paramagnetic properties make magnetic resonance imaging (MRI) a perfect candidate for the non-invasive estimation of iron content. Quantitative MRI (qMRI) techniques have revolutionized the field of MRI by providing biophysical parametric measurements of brain tissue that can be measured in standardized physical units and compared across subjects, sites, and time points. qMRI measurements have been linked to a variety of microstructural properties including iron. Specifically, iron is a major contributor to the longitudinal and transverse relaxation rates, and quantitative susceptibility mapping (QSM). In-vivo studies often use these qMRI measurements as a proxy for iron concentration. However, these methods have two major limitations. First, the sensitivity of qMRI measurements to the presence of iron is confounded by their sensitivity to the myelin content, as hinted by the strong MR contrast between white matter (WM) and gray matter (GM). In addition, non-invasive discrimination between different molecular forms of iron in the brain still remains a challenge.

Early works suggest that the physical state of brain iron can be revealed by the relaxivity of iron, defined as the dependency of MR relaxation rates on the iron concentration. It was shown that the iron relaxivity varies with the specific molecular form and environment in which the iron resides. Moreover, as the transverse and longitudinal relaxation rates are governed by different molecular and mesoscopic mechanisms 33, each of them has distinct relaxivity in the presence of paramagnetic substances. Until now, the phenomena of iron relaxivity was studied either in-vitro or post-mortem, as it requires quantification of the iron concentration.

Accordingly, a method for detecting iron-related entities in a region of interest, for example, the brain, using MRI signals, is suggested.

### SUMMARY OF THE INVENTION

Some aspects of the invention may be related to a method of detecting iron-related entities in a region of interest, comprising: receiving at least two Magnetic Resonance Imaging (MRI) signals from each of at least two different locations in the region of interest; calculating at least four MRI parameters from the MRI signals, and determining a presence of at least two different iron-related entities in the region of interest based on the at least four MRI parameters.

In some embodiments, the at least two different iron-related entities are selected from, iron-related compounds, loads of iron ions within a compound, spatial distributions of the iron-related compounds, and spatial structures of the iron-related compounds. In some embodiments, the at least two iron-related compounds are iron-binding proteins. In some embodiments, the iron-binding proteins are selected from: transferrin-bound iron, ferritin-bound iron, iron-sulfur protein, hemoglobin, hemosiderin, neuromelanin, magnetite, and myoglobulin. In some embodiments, the at least two different iron-related entities differ in the form of iron. In some embodiments, the form of iron is selected from, free iron, Fe²⁺, and Fe³⁺.

In some embodiments, the region of interest is selected from: a tissue, a laboratory phantom type sample, an in-vivo organ, and an ex-vivo organ.

In some embodiments, the method further comprising calculating the relative amount of each iron-related entity in the region of interest. In some embodiments, the method further comprising estimating the absolute amount of each iron-related entity in the region of interest. In some embodiments, the method further comprising determining locations of each iron-related entity in the region of interest.

In some embodiments, the method further comprising presenting the locations on an MRI scan from the region of interest or a model of the region of interest. In some embodiments, the method further comprising identifying different sub-regions in the region of interest, based on the determined locations. In some embodiments, identifying different sub-regions comprises differentiating between the sub-regions based on amounts of each iron-related entity in each sub-region.

In some embodiments, the method further comprising calculating interdependencies between the at least four MRI parameters and wherein determining the presence of the at least two different iron-related entities is based on the interdependencies.

In some embodiments, the at least four MRI parameters are selected to be dependent on iron relaxivity. In some embodiments, the at least four MRI parameters are quantitative MRI (qMRI) parameters. In some embodiments, the qMRI parameters are selected from, longitudinal relaxation rate (R1), transverse relaxation rates (R2, R2′, and R2*), Quantitative Susceptibility Mapping (QSM), diffusion, Magnetization Transfer (MT), and Chemical Exchange Saturation Transfer (CEST).

In some embodiments, further comprising determining a medical state of the region of interest based on the presence of the at least two different iron-related entities. In some embodiments, the medical state is a tumor. In some embodiments, the tumor is a brain tumor, for example, meningioma. In some embodiments, the method is performed without contrasting agent.

Some additional aspects of the invention are direct to a computer-based system comprising a processor configured to execute a method according to any one of the embodiments disclosed herein.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

to FIGS. 1A, 1B, 1C, 1D, 1E, and IF are graphs showing nonlimiting examples for assessing iron forms, in vitro, using R1 and R2*, according to some embodiments of the invention;

FIGS. 2A, 2B, 2C, and 2D are graphs showing nonlimiting examples for various qMRI parameters calculated from MRI signals received from different regions in a human brain according to some embodiments of the invention;

FIGS. 3A, 3B, 3C, and 3D include illustrations and graphs demonstrating nonlimiting examples for diagnosing meningioma brain tumors using R1-R2* slopes according to some embodiments of the invention;

FIGS. 4A, 4B, and 4C show the effect of iron concentration on different MR parameters according to some embodiments of the invention.

FIG. 5A is a block diagram, depicting a system for detecting iron-related entities in a region of interest according to some embodiments of the invention;

FIG. 5B is a block diagram, depicting a computing device which may be included in a system for detecting iron-related entities in a region of interest according to some embodiments of the invention;

FIG. 6 is a flowchart of a method of detecting iron-related entities in a region of interest according to some embodiments of the invention;

FIGS. 7A, 7B, and 7C are graphs showing the effect of iron concentration on different qMRI parameters according to some embodiment of the invention;

FIGS. 8A, 8B, 8C, 8D, 8E, and 8F are graphs showing the sensitivity of the iron relaxivity to the molecular types of the iron according to some embodiments of the invention;

FIGS. 9A, 9B, and 9C are graphs demonstrating validation of the sensitivity of the iron relaxivity and the R1-R2* slopes to the paramagnetic properties of transferrin according to some embodiments of the invention;

FIG. 10 is a column diagram showing the R1-R2* slopes for different ion-binding proteins at different fractions of liposomal, according to some embodiments of the invention;

FIGS. 11A and 11B are graphs showing the dependency of R1 on macromolecular tissue volume (MTV) for different lipids (PC and PC-SM) according to some embodiments of the invention;

FIG. 12 is a graph showing the dependency of R1 on R2* for different types of compositions comprising lipids and iron-binding protein; and

FIGS. 13A, 13B, 13C, and 13D are graphs showing the sensitivity of different qMRI parameters to the iron content in a mixture of the iron-binding proteins and lipids, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting to the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are outlined in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of the same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

Embodiments of the present invention disclose an MRI based method and a system for detecting iron-related entities in a region of interest. The region of interest may be any organ or tissue of a subject (e.g., either a human or animal) or a laboratory phantom. In some embodiments, the method is based on the detection of iron-relaxivity in MRI signals for different iron-related entities.

As used herein “an iron-related entity” may include any organic entity that is associated with iron in any kind of bound, including electrostatic bound. For example, the iron-related entities may be selected from, iron-related compounds, loads of iron ions within a compound, spatial distributions of the iron-related compounds, and spatial structures of the iron-related compounds. In some embodiments, the iron-related compounds are iron-binding proteins, for example, transferrin-bound iron, ferritin-bound iron, iron-sulfur protein, hemoglobin, hemosiderin, neuromelanin, magnetite, myoglobulin, and the like. In some embodiments, loads of iron ions within a compound refer to the amount of iron ions in a compound. For example, two ferritin proteins may differ in the amounts of iron ions bonded to the ferritin molecule. In some embodiments, spatial distributions of the iron-related compounds may refer to the distribution of iron ions in the three-dimensional (3D) structure of the molecule. In some embodiments, the spatial structures of the iron-related compounds may refer to the actual spatial morphology of the molecule, for example, protein aggregation or protein-protein complexes or protein-non protein complexes.

In some embodiments, two different iron-related entities may differ in the form of iron, for example, free iron, Fe²⁺, and Fe³⁺.

As used herein, “region of interest” refers to any region/volume where it is desired to have spatial information (image) of at least one form of iron/iron bound entities for research, diagnosis, and clinical purposes. Non-limiting examples of such regions are phantom (for research purposes), ex vivo organ (post mortem), in vivo organ, and the like. The in vivo organ may be, for example, the brain, kidney, liver, blood vessel, skeletal muscle, and the like.

For example, the region of interest may be selected from: a tissue, a laboratory phantom type sample, an in-vivo organ (e.g., the brain, liver, heart, kidney, etc.), and an ex-vivo organ (in an autopsy) and the like.

In some embodiments, from each location, at least two separate MRI signals may be received. In some embodiments, at least 5, 10, 15, and 20 signals (and any value in between) may be received from each location.

In some embodiments, MRI signals may be received from at least two different locations within the region of interest. In some embodiments, MRI signals may be received from more than 5, 10, 15, 20, 50, and 100 locations (and any value in between) within the region of interest.

In some embodiments, the MRI signals may be processed and at least one MRI parameter may be calculated from each signal. In some embodiments, the MRI parameters are dependent on the iron relaxivity of the iron ions in the iron-related entity. For example, the MRI parameters may be quantitative MRI (qMRI) parameters. Nonlimiting examples for qMRI parameters may include longitudinal relaxation rate (R1), transverse relaxation rates (R2, R2′, and R2*), Quantitative Susceptibility Mapping (QSM), diffusion, Magnetization Transfer (MT), Chemical Exchange Saturation Transfer (CEST) and the like.

In another example, the MRI parameters may be R1—weighted R2—weighted, R2*—weighted, R2′—weighted, QSM—weighted, QMT—weighted, CEST—weighted, Diffusion MRI-weighted and the like.

In some embodiments, the present invention is based on the finding that, where it is desired to spatially determine the level of specific forms of the iron/iron-related entities, it is possible to do so by determination of the interdependencies between at least two iron-related MRI parameters. This can give spatial information (imaging) of the levels of that specific iron form. It was found that each iron form/iron-related entity has a different linear relationship between its two MRI-relaxation values. Therefore, it may be possible to spatially distinguish between the different iron forms in the region of interest (e.g., an organ.).

This finding may be used to image specific forms of iron/iron-related entities in a region of interest for research, diagnostic, therapeutic treatment, and therapeutic monitoring purposes. The determination may be for imaging the iron form/iron bound entity itself, a determination that has diagnostic, therapeutic, and research implications for many diseases and pathological conditions. Nonlimiting examples of such diseases and pathological conditions are neurodegenerative disease, developmental impairment, cancer, stroke, infections, inflammatory diseases, and hemochromatosis.

The determination may also use the iron form/iron-related entity as the “imaging” or “contrast” agent of the region of interest—not for gaining information on the iron levels but for gaining information that is clinically important such as the presence, exact location, and size of a tumor—without the use of other (potentially damaging) contrast agents.

In a nonlimiting example, two qMRI parameters, the longitudinal and effective transverse relaxation rates, R1 and R2* were used for creating a biophysical model of their linear interdependency (the R1-R2* slope). This biophysical model suggested that two iron compounds displaying different iron relaxivities will also differ in their R1-R2* slopes. The hypothesis was proven on iron-containing phantom laboratory samples. The R1-R2* slopes increase the specificity of MRI to different iron compounds. When tested in a human brain it was shown that the in-vivo iron relaxivity model provides a new MRI contrast. This contrast may allow detecting of microstructural properties inaccessible by conventional qMRI approaches, based on gene enrichment analysis performed on resected tumor tissue. Furthermore, the in-vivo iron relaxivity contrast proves useful for enhancing the distinction between tumor tissue and non-pathological tissue. The specificity of this contrast to molecular iron forms was validated against histology. The in-vivo iron relaxivity allowed the prediction of the inhomogeneous distribution of iron-binding proteins due to aging and across the brain, and to reveal differences in iron hemostasis in meningiomal tissues.

Reference is now made to FIGS. 1A, 1B, 1C, 1D, 1E, and IF are graphs showing nonlimiting examples for assessing iron forms, in vitro, using R1 and R2*, according to some embodiments of the invention. The measurements were conducted on phantom samples made from different mixtures of proteins, iron-binding proteins, and/or lipids. FIGS. 1A and 1B show the dependency of R1 and R2* on the iron-binding protein concentration, tested in vitro, for the following different iron forms: unbound ferritin, liposomal-ferritin, Bovine Serum Albumin (BSA), ferritin mixture, unbound transferrin, and liposomal transferrin. Grey dots represent samples with varying liposomal fraction and iron concentrations.

FIG. 1C shows the ambiguity in R1 for different compounds. As shown R1 is higher for higher ferritin concentrations compared to lower ferritin concentrations, and is also higher for ferritin compared to transferrin.

FIG. 1D shows the iron relaxivity: consistent when computed over higher or lower ferritin concentrations, and is consistently different from the iron relaxivity of transferrin regardless of the concentration.

FIG. 1E shows the dependency of R1 on R2* for different iron forms. The R1-R2* slopes are different for each iron-related entity. The iron-related entities are BSA+ferritin, transferrin, liposomal (PC-SM)+transferrin, PC-SM+ferritin, and ferritin. As clearly shown in the graph each entity (e.g., a combination of iron-binding proteins with or without lipids (e.g., liposomal) has a specific R1-R2* slope which can be used to identify the entity in a region of interest.

FIG. 1E shows the R1-R2* slopes for the iron-related entities of FIG. 1E. The illustrated boxes extend to the 95% confidence interval of the linear fit. The grey dots represent the prediction of the theoretical model (based on the ratio between the iron relaxivities of R1 and R2*). As shown the measured R1-R2* slopes are almost identical to the theoretical model.

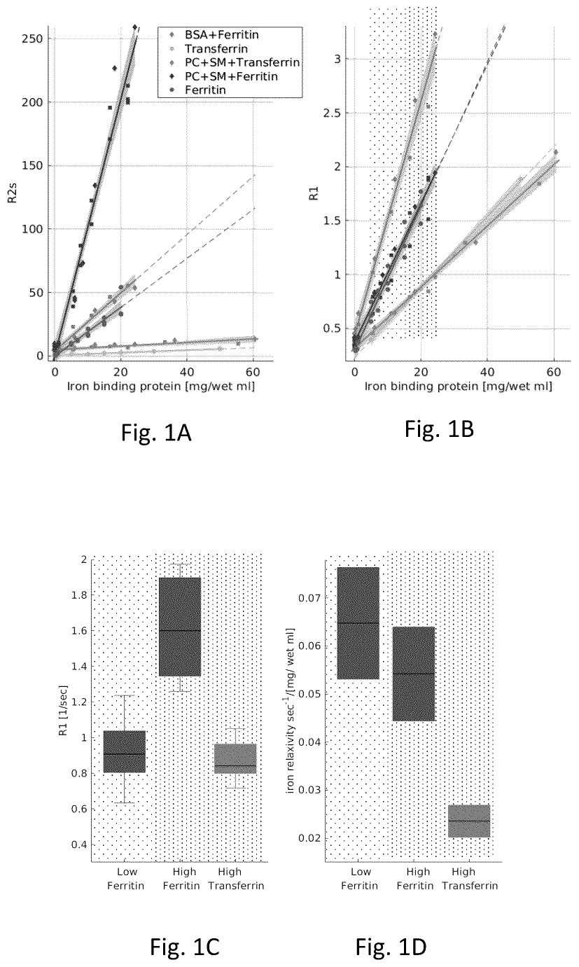

Reference is now made to FIGS. 2A, 2B, 2C, and 2D which are graphs showing nonlimiting examples for various qMRI parameters calculated from MRI signals received from different regions in a human brain according to some embodiments of the invention. FIG. 2A shows the dependency of R1 on R2* in four brain regions of a single subject. R2* and R1 were binned (dots are the median; shaded area is the MAD), and a linear fit was calculated. The slopes of the linear fit represent the dependency of R1 on R2* (R1-R2* slope) and vary across the different brain regions. FIG. 2A shows the R1-R2* slopes across the brain. On the left side, the boxes show the reliability of the method in different brain regions (left hemisphere) as observed by the variation in the R1-R2* slopes across young subjects (N=27). The 25th, 50^{th}, and 75th percentiles and extreme data points are shown for each box. On the right side, the graph shows the contrast of the R1-R2* slopes across the brain. The different grey levels distributions represent the values of the R1-R2* slopes in sub-cortical, white-matter, and cortical brain regions respectively.

FIGS. 2C and 2D show similar analysis conducted for R1 and R2* values, in which the gray-matter vs. white-matter contrast is much more dominant.

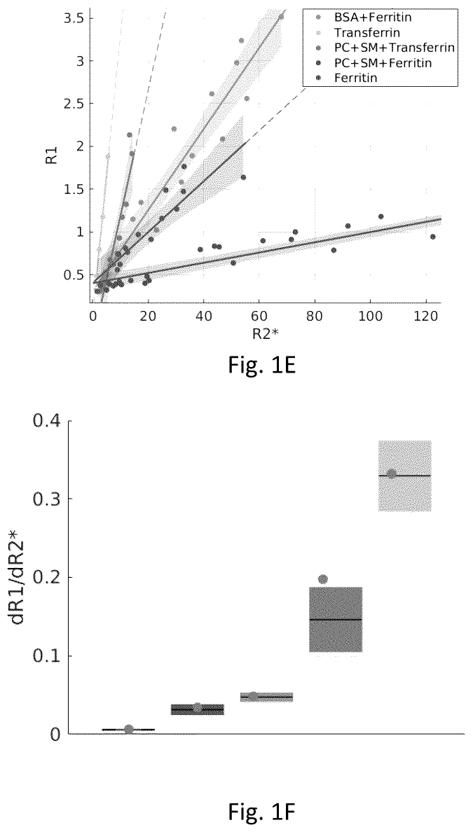

Reference is now made to FIGS. 3A, 3B, 3C, and 3D which include illustrations and graphs demonstrating nonlimiting examples for diagnosing meningioma brain tumors using R1-R2* slopes according to some embodiments of the invention. FIG. 3A are MRI scans showing tumor segmentation, R1 and R2* maps in a representative subject. The tumor segmentation was done manually by a neurosurgeon and is overlaid on a T1-weighted MRI scan. As shown in FIG. 1A the area containing the tumor is clearly shown on the R1 and R2* maps. FIG. 3B shows a dependency of R1 on R2* in the white matter, gray matter, and tumor tissue of a single subject. Tumor tissue exhibits a distinct slope relative to non-pathological tissue.

FIG. 3C shows the contrast between the white matter, gray matter, and tumor tissue across 16 subjects. On the left side: each box shows the variation in R1-R2* slopes across subjects for a different tissue. The 25th, 50^{th}, and 75th percentiles and extreme data points are shown. d-values represent the effect size (Cohen's d) of the differences between tissues. On the right side: the contrast of the R1-R2* slopes across different tissues is presented. FIG. 3D shows a similar analysis conducted for Gd-enhanced MRI contrast.

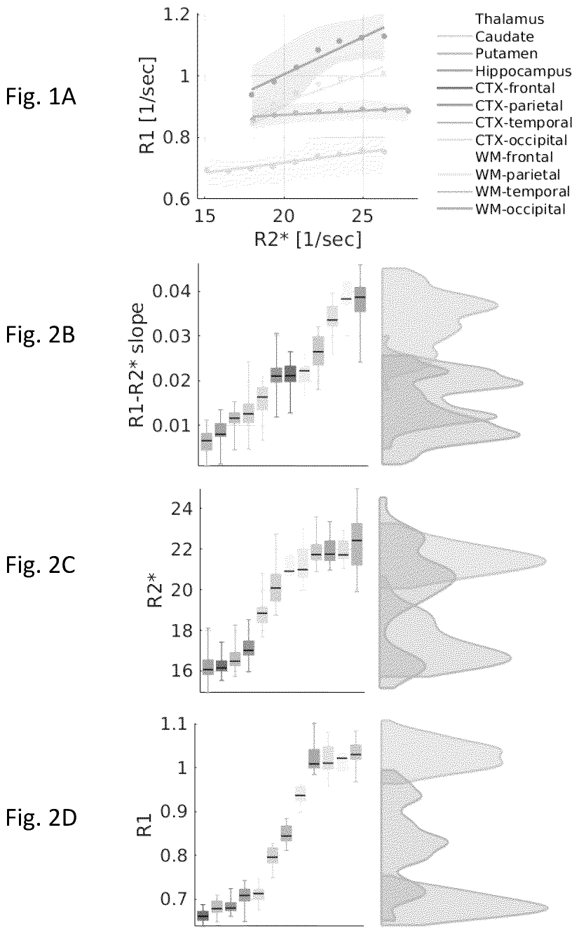

Reference is now made to FIGS. 4A, 4B, and 4C show the effect of iron concentration on different qMRI parameters according to some embodiments of the invention. FIG. 4A shows a comparison between tumor tissue with low (<1) and high (>1) transferrin to ferritin ratio (Tf/Fer) estimated using western-blot following surgical resection of the tissue. As clearly shown in FIG. 4A the R1-R2* slopes were higher for high Tf/Fer ratio, therefore can be used to distinguish between areas with high and low transferrin to ferritin ratio.

FIG. 4B is a graph showing the transferrin/iron ratio (post-mortem, from the literature) is correlated with the R1-R2* slopes measured in vivo across younger (<64) and older subjects (>64) in 11 brain regions.

FIG. 4C is a graphical representation of a fully-constrained model for predicting the fractions of iron-binding proteins in vivo in the human brain. The measured R1-R2* slope in each brain area was modeled as a weighted sum of the R1-R2* slopes of transferrin (Tf) and ferritin (Fer) (estimated in liposomal phantoms). The Tf and Fer fractions sum to one. In some embodiments, the model allows predicting the transferrin fraction (Tf/(Tf+Fer), y-axis) for younger (<64) and older subjects in 11 brain regions. There are no free parameters. The x-axis is the Tf fraction measured post-mortem. The MAE is the Mean Absolute Error.

As clearly shown in the nonlimiting examples given hereinabove qMRI parameters can be used to determine the presence of at least two different iron-binding proteins, determine quantities of these iron-related proteins and diagnose abnormalities (e.g., a tumor) in a region of interest based on these at least two different iron-binding proteins. As would be understood by one skilled in the art, similar methodologies and methods can be applied using other MR parameters, for example, R1—weighted R2—weighted, R2*—weighted, R2′—weighted, QSM—weighted, QMT—weighted, CEST—weighted, Diffusion MRI—weighted. As further be understood by one skilled in the art, the iron-binding proteins were given as examples only, and any iron-related entity showing iron relaxation in an MRI signal can be detected using similar methods.

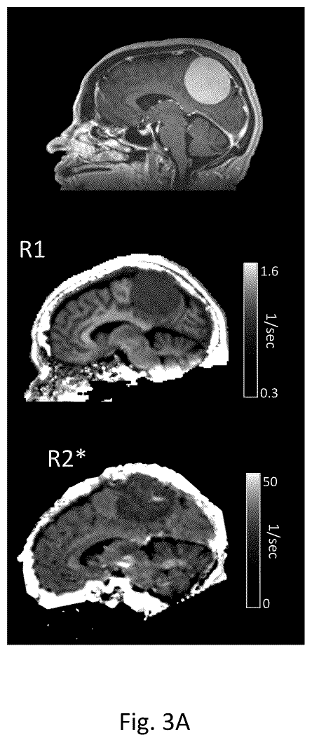

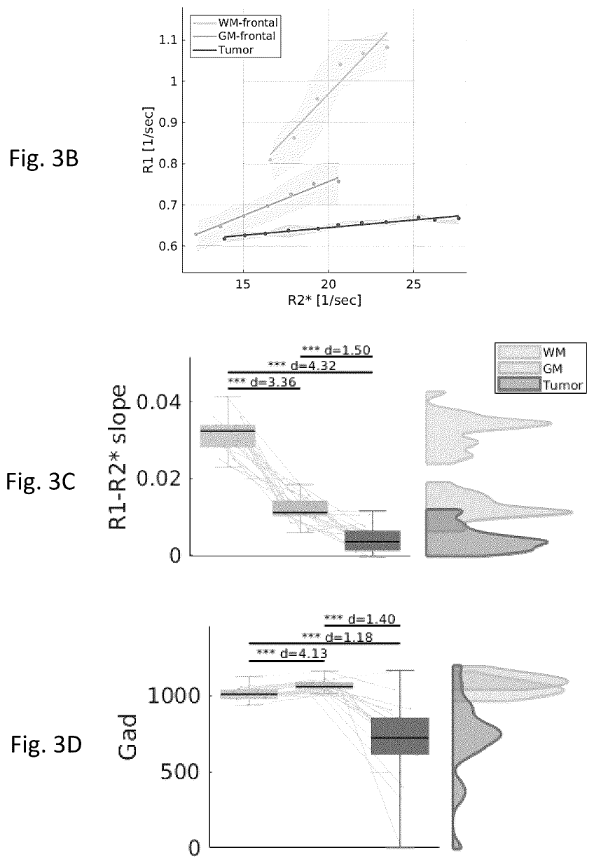

Reference is now made to FIG. 5A which is a block diagram of a system for detecting iron-related entities in a region of interest according to some embodiments of the invention. A system 100 may include, at least one computing device 10 discussed hereinbelow, with respect to FIG. 5B and an MRI unit 20. MRI unit 20 may be any MRI unit known in the art, for example, 3 Tesla magnetic field. MRI unit 20 may be configured to subject a region of interest 30 to strong magnetic fields and transmit and receive RF signals from region of interest 30 to produce MRI signals. The MRI signals may be processed and analyzed by at least one computing device 10 according to embodiments of the present invention discussed herein.

Reference is now made to FIG. 5B, which is a block diagram depicting a computing device, which may be included within an embodiment of a system for detecting iron-related entities in a region of interest, according to some embodiments.

Computing device 10 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 10 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling, or otherwise managing the operation of computing device 10, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random Access Memory (RAM), a read-only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task, or script. Executable code 5 may be executed by processor or controller 2 possibly under the control of operating system 3. For example, executable code 5 may be an application that may detect iron-related entities in a region of interest as further described herein. Although, for the sake of clarity, a single item of executable code 5 is shown in FIG. 5B, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a microcontroller or chip as known in the art, a hard disk drive, a CD-Recordable (CD-R) drive, a Blu-ray disk (BD), a universal serial bus (USB) device or other suitable removable and/or fixed storage unit. For example, MRI parameters of different iron-related entities associated with different medical conditions may be stored in storage system 6 and may be loaded from the storage system 6 into memory 4 where it may be processed by processor or controller 2. In some embodiments, some of the components shown in FIG. 5B may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components, or systems, e.g., a detachable keyboard or keypad, a mouse, and the like. Output devices 8 may include one or more (possibly detachable) displays or monitors, speakers, and/or any other suitable output devices. Any applicable input/output (I/O) devices may be connected to Computing device 1 as shown by blocks 7 and 8. For example, a wired or wireless network interface card (NIC), a universal serial bus (USB) device, or an external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

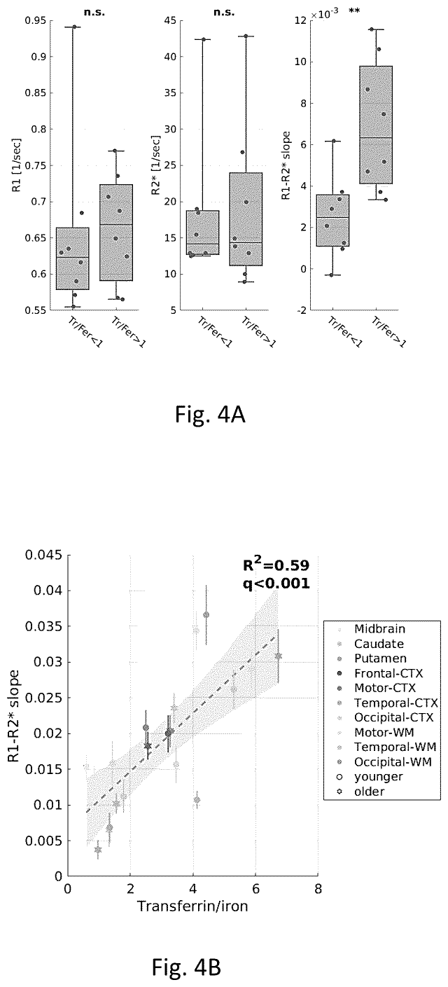

Reference is now made to FIG. 6 which is a flowchart of a method of detecting iron-related entities in a region of interest according to some embodiments of the invention. The method of FIG. 6 may be executed by processor 2 of computing device 10 according to a code (e.g., executable code 5) stored in memory 4, or by any other suitable processor. In step 610, at least two MRI signals may be received from each of at least two different locations in the region of interest. In some embodiments, the MRI signals may be received directly from an MRI unit or from a database (e.g., storage system 6) storing MRI signals.

In some embodiments, the region of interest is selected from tissue, a laboratory phantom type sample, an in-vivo organ, and an ex-vivo organ. In some embodiments, a minimum of four MRI signals may be received, 2 signals from each one of the two locations. In some embodiments, more than two signals may be received from each one of the two locations. In some embodiments, signals may be received from more than two locations. In a nonlimiting example, 4-10 signals may be received from each one of 20-10000 locations.

In step 620, at least four MRI parameters may be calculated from the MRI signals. In some embodiments, the MRI parameters may be qMRI parameters. Additionally or alternatively, the MRI parameters may be R1—weighted R2—weighted, R2*—weighted, R2′—weighted, QSM—weighted, QMT—weighted, CEST—weighted, Diffusion MRI—weighted. In some embodiments, the at least four MRI parameters are selected to be dependent on iron relaxivity. In some embodiments, the qMRI parameters are selected from, longitudinal relaxation rate (R1), transverse relaxation rates (R2, R2′, and R2*), Quantitative Susceptibility Mapping (QSM), diffusion, Magnetization Transfer (MT), Chemical Exchange Saturation Transfer (CEST) and the like.

In step 630, a presence of at least two different iron-related entities in the region of interest may be determined based on the at least four MRI parameters. In some embodiments, the at least two different iron-related entities are selected from, iron-related compounds, loads of iron ions within a compound, spatial distributions of the iron-related compounds, and spatial structures of the iron-related compounds. In some nonlimiting examples, the at least two iron-related compounds are iron-binding proteins, selected from transferrin-bound iron, ferritin-bound iron, iron-sulfur protein, hemoglobin, hemosiderin, neuromelanin, magnetite, and myoglobulin. In some embodiments, the at least two different iron-related entities differ in the form of iron, wherein the form of iron is selected from, free iron, Fe²⁺, and Fe³⁺.

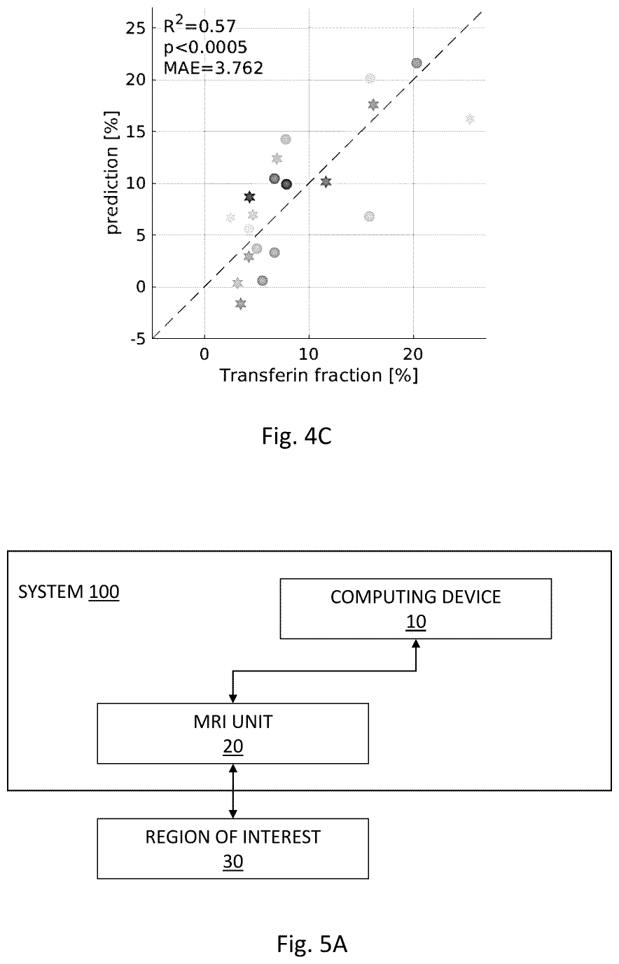

Nonlimiting examples for determining the presence of iron-binding proteins in regions of interest are given with respect to FIGS. 1 to 4 and further with respect to FIGS. 7 to 13. As should be understood by one skilled in the art similar methods (e.g., slops calculations and the correlation between the slops to the presence/amount of iron-related entities in the region of interest) are applicable to other MRI parameters and to other iron-related entities

In some embodiments, the method may further include calculating the relative amount and/or the absolute amount of each iron-related entity in the region of interest. For example, processor 2 may calculate the relative amount and/or the absolute amount of each iron-related entity based on the values of the MRI parameters (e.g., R1 and R2*) in each location in the region of interest. In some embodiments, the method may further include calculating interdependencies between the at least four MRI parameters and wherein determining the presence of the at least two different iron-related entities is based on the interdependencies. As used herein the term “interdependencies” refers to a description of one iron-related MRI value as functions of another iron-related MR value. This function can be linear and non-linear. Examples are: slope for the linear relationship and higher-order coefficients for non-linear functions. Some nonlimiting examples for such interdependencies are R1-R2* slop, R1-MT slop, and the like.

In some embodiments, the method may include determining locations of each iron-related entity in the region of interest. for example, as illustrated and discussed with respect to FIGS. 3A-3D, which show the R1/R2* contrast of a tumor (e.g., sub region) in the brain and the calculation of the R1-R2* slop which is indicative of the amount of iron-binding proteins in each sub-region. In some embodiments, the locations may be presented on an MRI scan from the region of interest or a model of the region of interest, for example, as shown in FIG. 3A. In some embodiments, the method may include identifying different sub-regions (e.g., a tumor) in the region of interest (e.g., the brain), based on the determined locations. In some embodiments, identifying different sub-regions comprises differentiating between the sub-regions based on amounts of each iron-related entity in each sub-region, according to any method disclosed herein.

In some embodiments, the method may include determining the medical state of the region of interest-based on the presence of the at least two different iron-related entities. For example, the method may allow detecting a tumor, for example, a tumor in the brain without the need to use contrast enhancers (contrasting agents). Nonlimiting example for brain tumor is meningioma.

### Additional Experimental Results

Reference is now made to FIGS. 7A, 7B, and 7C which are graphs showing the effect of iron concentration on different qMRI parameters according to some embodiment of the invention. FIGS. 7A to 1C show the potential to use the computation of the iron relaxivity as the dependency of R1 and R2* in detecting the presence and the concentration of iron-binding proteins. As shown in FIGS. 7A to 7C, different forms of iron have different relaxivities. However, different proteins bind different amounts of iron. For example, ferritin binds two orders of magnitude more iron than transferrin. In some embodiments, it was verified that the relaxivity changes with the molecular type of iron, even when taking the discrepancies in iron-binding into account. In some embodiments, these properties can be useful in estimating the iron concentration for ferritin and transferrin and testing whether this can explain their different iron relaxivities.

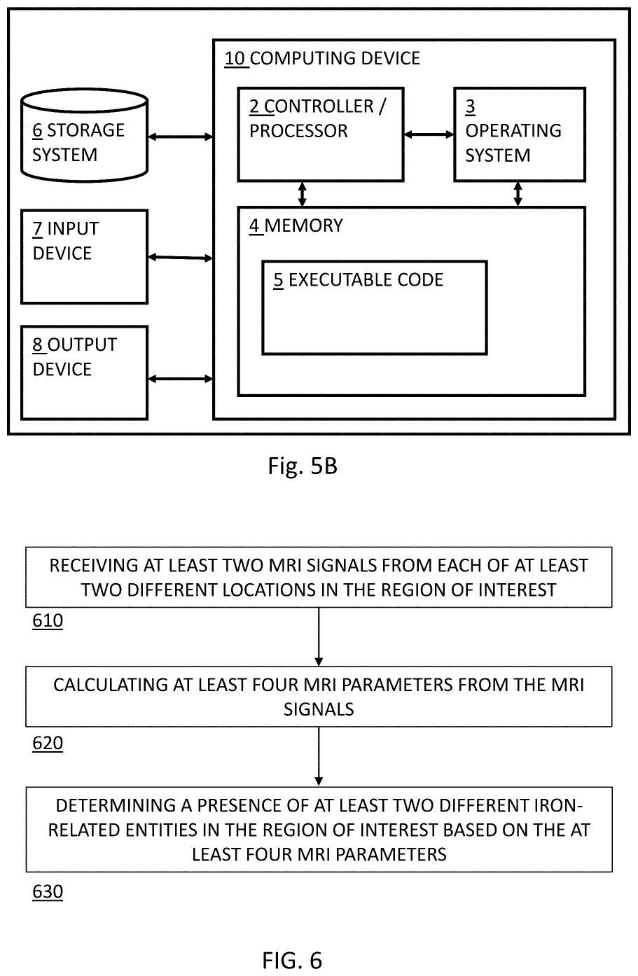

Reference is now made to FIGS. 8A, 8B, 8C, 8D, 8E, and 8F which are graphs showing the sensitivity of the iron relaxivity to the molecular types of the iron according to some embodiments of the invention. In some embodiments, when computing the iron relaxivity as the dependency of relaxation rates on the iron concentrations it was surprisingly found that find different iron forms have distinct slopes (FIGS. 2A and 2B). The graphs for FIGS. 2A and 2B were calculated using ANCOVA (analysis of variance (ANOVA) and regression) software pack * (p<10-40) for the interaction of iron type*iron concentration. Furthermore, the findings were verified in by calculating the iron concentrations, as discussed in FIGS. 2C and 2D. It was found that R1 was higher for higher ferritin concentrations compared to lower ferritin concentrations but was also higher for ferritin compared to transferrin. This ambiguity was resolved by the iron relaxivity, also when evaluated based on the iron concentration (rather than the protein concentration). The iron relaxivity was consistent when computed over higher or lower ferritin concentrations, and was consistently different from the iron relaxivity of transferrin regardless of the concentration, as shown in FIG. 2D.

To further stress the sensitivity of R2* and R1 to the type and concentration of iron, the relaxivity of liposomal ferrous iron (Fe2+) and iron bound to liposomal transferrin were compared, as shown in the graphs of FIGS. 8E and 8F. Unlike ferritin and transferrin, these two iron forms have relatively similar iron concentrations. It was surprisingly found that different concentrations produce different relaxation rates (p<10⁻⁴ tested with ANCOVA for the interaction of iron type*iron concentration). Therefore, R1 and R2* change both with the concentration of iron and with the molecular form of iron.

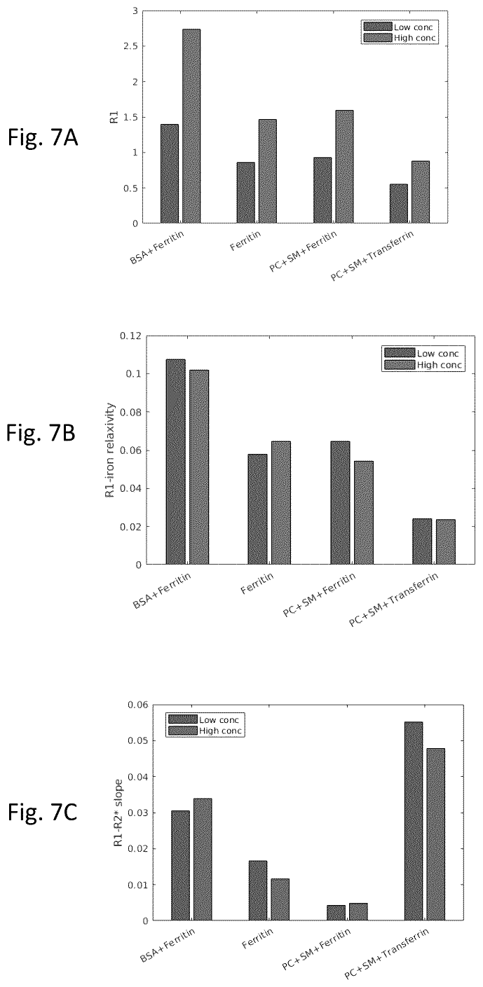

Reference is now made to FIGS. 9A, 9B, and 9C which demonstrate validation of the sensitivity of the iron relaxivity and the R1-R2* slopes to the paramagnetic properties of transferrin according to some embodiments of the invention. FIGS. 9A and 0B show the dependency of R1 and R2* on the iron-binding protein concentration for three different iron states: liposomal-ferritin, liposomal transferrin, and liposomal apo-Transferrin. Colored dots represent samples with varying liposomal fraction and iron concentrations. Transferrin that is not bound to iron (apoTransferrin) has a different iron relaxivity compared to iron-bound transferrin. FIG. 9C shows the dependency of R1 on R2* for liposomal transferrin and liposomal apoTransferrin. The R1-R2* slopes are different when transferrin is bound to iron compared to apoTransferrin, validating the specificity of this measurement to the paramagnetic properties of transferrin.

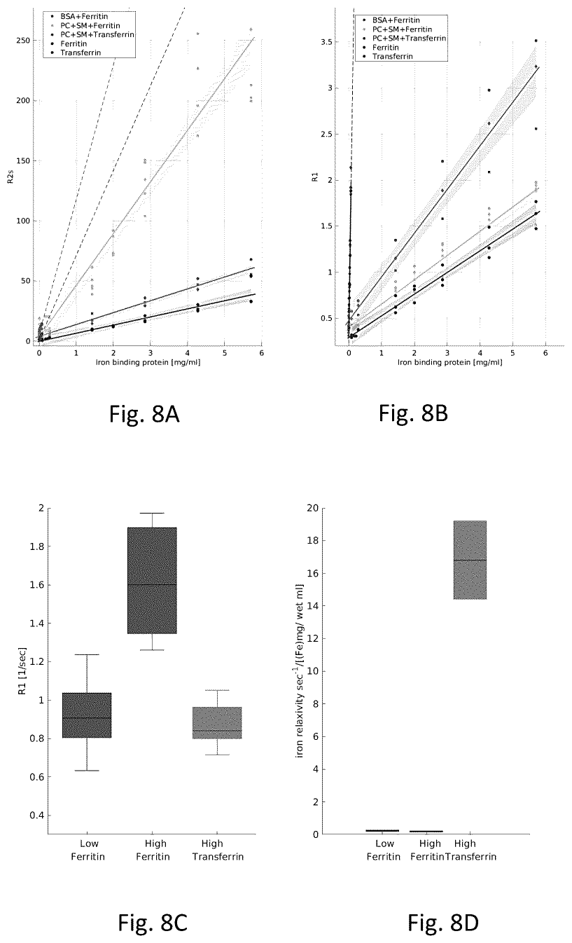

Reference is now made to FIG. 10 which is a column diagram showing the R1-R2* slopes for different ion-binding proteins at different fractions of liposomal, according to some embodiments of the invention. The differences in R1-R2* slopes between iron forms are greater than the differences within each iron form for the different liposomal/protein fractions. R1 and R2* are known to be sensitive to the myelin content. To test the effect of the myelin fraction on the iron relaxivity, the liposomal fractions were varied in phantom experiments. The in-vivo iron relaxivity measured with the R1-R2* slope was stable for the different liposomal fractions.

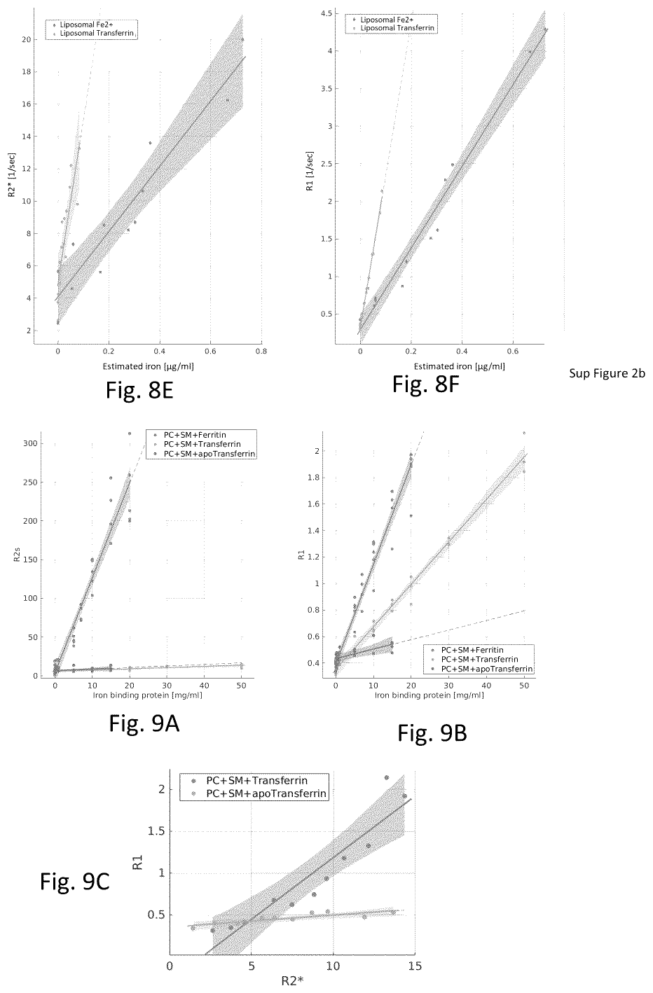

Reference is now made to FIGS. 11A and 11B which show the dependency of R1 on macromolecular tissue volume (MTV) for different lipids (PC and PC-SM) according to some embodiments. FIG. 11A shows the dependency of R1 on MTV for two different lipids (PC and PC-SM) mixed with the same form of iron (Fe2+). FIG. 11B shows the dependency of R1 on MTV for two different lipids (PC and PC-SM) mixed with the same form of iron (Ferritin). As shown, R1-MTV slopes are different for two types of lipids (phosphatidylcholine and phosphatidylcholine-sphingomyelin) mixed with irons. In the presence of ferritin, the difference between the R1-MTV slopes of the two lipids is smaller (FIG. 5B). The R1-R2* slopes are insensitive to the lipid composition.

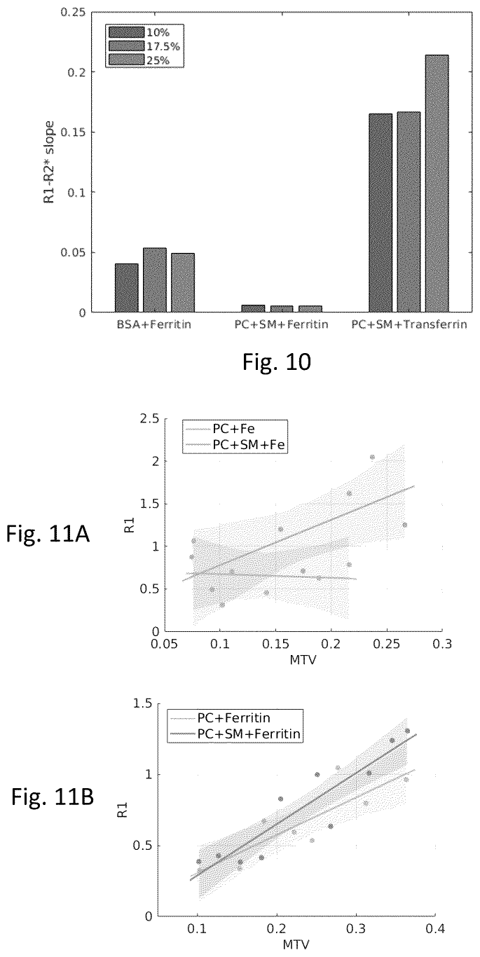

Reference is now made to FIG. 12 which shows the dependency of R1 on R2* for different types of lipid-iron binding protein compositions. The samples include mixtures of three different types of lipids (PC, PC-SM, and PC-Chol) with ferritin and for transferrin sample mixed with PC-SM. The R1-R2* slopes are similar for the different lipid types, and the main difference is between the ferritin samples to the transferrin sample. Accordingly, the type of lipid has a negatable effect on the relaxivity on iron ions and the respective qMRI parameters.

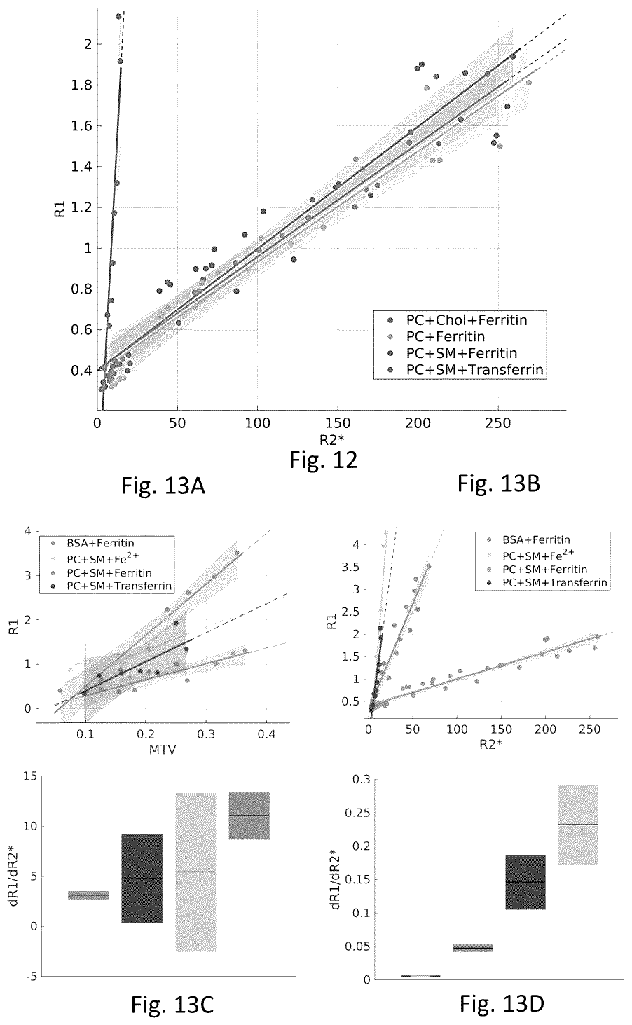

Reference is now made to FIGS. 13A, 13B, 13C, and 13D which show the sensitivity of different qMRI parameters to the iron content in a mixture of the iron-binding proteins and lipids, according to some embodiments of the invention. The qMRI parameters are R1-MTV slops (FIGS. 13A and 13C) and R-R2* slops (FIGS. 13B and 13D). FIG. 1A shows the dependency of R1 on MTV for different iron-related entities: liposomal (PC-SM)-ferritin, Bovine Serum Albumin (BSA), and ferritin mixture, liposomal (PC-SM) transferrin and liposomal (PC-SM) Fe2+. FIG. 2B shows the dependency of R1 on R2* for the same different iron forms: FIGS. 13C and 13D show boxplots displaying the R1-MTV and the R1-R2* slopes. Box edges represent the 95% confidence interval for the slopes. When comparing the R1-MTV slopes, to the R1-R2* slopes, it was found that the latter provided better distinction between iron forms.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of detecting iron-related entities in a region of interest, comprising:
receiving, at least two Magnetic Resonance Imaging (MRI) signals from each of at least two different locations in the region of interest;
calculating at least four MRI parameters from the MRI signals; and
determining a presence of at least two different iron-related entities in the region of interest-based on the at least four MRI parameters.

2. The method of claim 1, wherein the at least two different iron-related entities are selected from, iron-related compounds, loads of iron ions within a compound, spatial distributions of the iron-related compounds, and spatial structures of the iron-related compounds.

3. The method according to claim 2, wherein the at least two iron-related compounds are iron-binding proteins.

4. The method of claim 3, wherein the iron-binding proteins are selected from: transferrin-bound iron, ferritin-bound iron, iron-sulfur protein, hemoglobin, hemosiderin, neuromelanin, magnetite, and myoglobulin.

5. The method according to claim 1, wherein the at least two different iron-related entities differ in the form of iron.

6. The method of claim 5, wherein the form of iron is selected from, free iron, Fe²⁺, and Fe³⁺.

7. The method according to claim 1, wherein the region of interest is selected from: a tissue, a laboratory phantom type sample, an in-vivo organ, and an ex-vivo organ.

8. The method according to claim 1, further comprising calculating the relative amount of each iron-related entity in the region of interest.

9. The method according to claim 1, further comprising estimating the absolute amount of each iron-related entity in the region of interest.

10. The method according to claim 1, further comprising determining locations of each iron-related entity in the region of interest.

11. The method of claim 10, further comprising presenting the locations on an MRI scan from the region of interest or a model of the region of interest.

12. The method according to claim 10, further comprising identifying different sub-regions in the region of interest, based on the determined locations.

13. The method of claim 12, wherein identifying different sub-regions comprises differentiating between the sub-regions based on amounts of each iron-related entity in each sub-region.

14. The method according to claim 1, further comprising calculating interdependencies between the at least four MRI parameters and wherein determining the presence of the at least two different iron-related entities is based on the interdependencies.

15. The method according to claim 1, wherein the at least four MRI parameters are selected to be dependent on iron relaxivity.

16. The method according to claim 1, wherein the at least four MRI parameters are quantitative MRI (qMRI) parameters, and wherein the qMRI parameters are selected from, longitudinal relaxation rate (R1), transverse relaxation rates (R2, R2′, and R2*), Quantitative Susceptibility Mapping (QSM), diffusion, Magnetization Transfer (MT), and Chemical Exchange Saturation Transfer (CEST).

17. (canceled)

18. The method according to claim 1, further comprising determining a medical state of the region of interest based on the presence of the at least two different iron-related entities.

19. The method of claim 18, wherein the medical state is a tumor.

20. The method of claim 19, wherein the tumor is a brain tumor.

21. (canceled)

22. (canceled)

23. A computer-based system comprising:
a processor; and
a memory storing thereon instructions to be executed by the processor, wherein the instructions comprises:
receiving, form an MRI unit, at least two Magnetic Resonance Imaging (MRI) signals from each of at least two different locations in the region of interest;
calculating at least four MRI parameters from the MRI signals; and
determining a presence of at least two different iron-related entities in the region of interest based on the at least four MRI parameters.
